SEQUENCE LISTING (6 sequences)

SEQ ID NO: 1 - DNA, 27 bp, Escherichia coli
ccgggatccaggaaggcgcgctgtccg

SEQ ID NO: 2 - DNA, 28 bp, Escherichia coli
aactcgtttcattttgatttcgtcctgg

SEQ ID NO: 3 - DNA, 30 bp, Escherichia coli
gacgaaatcaaaatgaaacgagttattacc

SEQ ID NO: 4 - DNA, 31 bp, Escherichia coli
gaatttgtcgatttgataaacaaaagtcacg

SEQ ID NO: 5 - DNA, 29 bp, Escherichia coli
tttgtttatcaaatcgacaaattccgttc

SEQ ID NO: 6 - DNA, 28 bp, Escherichia coli
ccgctcgagtcacgcttcagacaggttg

(19) United States
(12) Patent Application Publication
(10) Pub. No.: US 2016/0015797 A1
(43) Pub. Date: Jan. 21, 2016

(54) VACCINE FOR UTI WITH TRUNCATED FORM OF FLAGELLIN (FliC) FROM ENTEROAGGREGATIVE ESCHERICHIA COLI FUSED WITH FimH PROTEIN

(71) Applicant: Pasteur Institute of Iran (IPI), Tehran (IR)

(72) Inventor: Saeid Bouzari, Tehran (IR)

(21) Appl. No.: 14/806,667

(22) Filed: Jul. 23, 2015

(30) Foreign Application Priority Data
Aug. 6, 2014 (IR) 13935014000300514

(51) Int. Cl. A61K 39/108

(52) U.S. Cl. CPC A61K 39/0258

(57) ABSTRACT

The embodiments herein discloses a vaccine against urinary tract infection (UTI). The flagellin (FliC) of enteroaggregative Escherichia coli is fused to FimH derived from uropathogenic Escherichia coli. The interaction of FliC and FimH with Toll-like receptor 5 (TLR-5) is analyzed in silico by docking protocols. The fused protein obtained after docking studies are subjected to cloning and expression in a vector. The recombinant vaccine expressed by the vector is purified. The recombinant vaccine has a size of 1200 bp. The ability of the recombinant vaccine FliCA-FimH-FliCB and the truncated form is analyzed by immunizing the mice. The result illustrate that the truncated forms are capable of inducing T helper 1 and T helper 2 cell response. It is also illustrated that the fusion vaccine induces a strong cellular and humoral immune response.

### CROSS-REFERENCE TO RELATED APPLICATIONS

This application claims the benefit of Iranian Patent application Ser. No. 139350140003005145 filed on Aug. 6, 2014, which is hereby incorporated by reference.

### BACKGROUND

1. Technical field

The embodiments herein generally relates to an immunogenic composition as a vaccine and a method for treating infections caused by gram negative bacteria. The embodiments herein particularly relate to a method of synthesizing truncated proteins for treating infections caused by gram negative bacteria. The embodiments herein also relates to synthesis of a vaccine comprising truncated for of flagellin fused to FimH of uropathogenic E. coli against urinary tract infections.

2. Description of the Related Art

Urinary tract infection (UTI) is also known as acute cystitis. UTI is an infection that affects part of the urinary tract. The UTI occurring in the lower urinary tract is known as a simple cystitis (a bladder infection). When the UTI occurs in upper urinary tract it is known as pyelonephritis (a kidney infection). Symptoms from a lower urinary tract infectin includes painful urination, frequent urination, urge to urinate, fever, flank pain and a painful burning sensation in the urethra. The main agent causing UTI is Escherichia coli, though bacteria viruses and fungi are also reported to cause UTI.

Generally urinary tract infection (UTI) is easily treated with a short course of antibiotics, although resistance to many of the antibiotics used to treat this condition is increasing. In complicated cases a longer course of intravenous antibiotics are needed.

Commonly used drugs specifically antibiotics for treating urinary tract infection are: ciprofloxacin, fosfomycin, levofloxacin, nitrofurantoin, sulfamethoxazole with trimethoprim.

There are many side effects of antibiotics used to treat urinary tract infection (UTI) and bladed infection include nausea, diarrhea, dizziness, light headedness, or trouble in sleeping. Other severe or less common side effects include fever, persistent sore throat, eye pain, vision changes, mood changes, easy bruising/bleeding, pain, numbness, burning sensation, stomach upset, yellowing skin, bloody urine, abdominal pain, chills, shortage of breath, fast heart beat, seizures and weakness.

Flagellin is a globular protein that arranges itself in a hollow cylinder to form the filament in a bacterial flagellum. The molecular weight of flagellin is 30,000 to 60, 000 daltons. Flagellin is the principle substituent of bacterial flagellum and is present in large amounts on nearly all flagellated bacteria.

The structure of flagellin is responsible for the helical shape of the flagellar filament, which is important for its proper function.

The N and C terminal of flagellin are responsible to form the inner core of the flagellin protein. The N and C terminal are also responsible for flagellin's ability to polymerize into a filament. The terminal part of flagellin protein is quite similar among all bacterial flagellin whereas the central portion is widely variable.

Flagellin (FliC) is the major component of the flagella of Gram negative bacteria. Flagellin is a potent trigger of innate immune responses in a number of eukaryotic cells and organisms, including mammals.

Flagellin (FliC-EAEC) is a major bacterial surface protein of EAEC, which causes interleukin (IL-8) release from several epithelial cell lines. The host responses to flagellins from E. coli are mediated by Toll-like receptor 5 (TLR-5), which signals through nuclear factor kappa B (NF-κB) to induce transcription of pro-inflammatory cytokines. Based on the pathogenic mechanism of FliC-EAEC and its ability to activate innate immunity the flagellin and its truncated forms are considered as a potent new adjuvant in vaccine formulations.

Urinary tract infections (UTI's) are among the most common bacterial infections worldwide. At least 80% of UTI infections are caused by uropathogenic Escherichia coli (UPEC). Increasing prevalence of antibiotic resistant UPEC strains are raising concerns. The development of an effective and safe vaccine against UPEC is highly desirable.

The adhesions are cell-surface components or appendages of bacteria that facilitate adhesion or adherence to other cells or to surfaces. Adhesins are a type of virulence factor. The best characterized bacterial adhesion is the type 1 fimbrial FimH adhesion. This adhesion is responsible for D-mannose sensitive adhesion. The bacterium synthesizes a precursor protein consisting of 300 amino acids then processes the protein by removing several signal peptides ultimately leaving a 279 amino acid protein. Mature FimH is displayed on the bacterial surface as a component of the type 1 fimbirial organelle FimH is folded into two domains. The N terminal adhesive domain plays the main role in surface recognition while the C-terminal domain is responsible for organelle integration. A tetrapeptide loop links the two domains. Additionally a carbohydrate binding pocket has been identified at the tip of the N-terminal adhesive domain.

The FimH is one of the most virulence factors of uropathogenic Escherichia coli (UPEC). Because of the critical role of adherence during infection, FimH is a prime target for UPEC vaccines.

Hence there is a need for synthesizing a vaccine against Escherichia coli causing urinary tract infection (UTI). Also there is a need for a vaccine which enhances the immune response naturally and provides resistance against Escherichia coli causing urinary tract infection (UTI). Further there is a need for a vaccine which does not have side effect on the recipient's immune response.

The above mentioned shortcomings, disadvantages and problems are addressed herein and which will be understood by reading and studying the following specification.

### OBJECTIVES OF THE EMBODIMENTS

The primary objective of the embodiment herein is to synthesize a recombinant vaccine against urinary tract infection consisting of two truncated forms of flagellin (FliC) protein of enteroaggregative Escherichia coli placed at N and C termini of FimH protein derived from uropathogenic Escherichia coli.

Another object of the embodiment herein is to synthesize a recombinant vaccine using flagellin from enteroaggregative Escherichia coli as an adjuvant.

Yet another object of the embodiment herein is to provide a truncated vaccine against urinary tract infection consisting of 79-117 and 477-508 amino acids from flagellin as an adjuvant.

Yet another object of the embodiment herein is to conduct in silico studies to obtain a truncated form of recombinant vaccine against urinary tract infection prior conducting in vitro construction of vaccine.

Yet another object of the embodiment herein is to synthesize a recombinant vaccine using flagellin for inducing the adaptive immune response in the recipients against urinary tract infection.

Yet another object of the embodiment herein to synthesize a recombinant vaccine using flagellin (FliC) protein and FimH protein for eliciting the humoral immune response in the recipients against urinary tract infection.

These objects and the other advantages of the embodiments herein will become readily apparent from the following detailed description taken in conjunction with the accompanying drawings.

### SUMMARY

The various embodiments herein provide a recombinant vaccine against urinary tract infection consisting of two truncated forms of flagellin (FliC) protein of enteroaggregative Escherichia coli placed at N and C terminal of FimH protein derived from uropathogenic Escherichia coli. The recombinant vaccine is synthesized using flagellin from enteroaggregative Escherichia coli as an adjuvant. The vaccine induces adaptive immune response and elicits humoral immunity in the recipients against urinary tract infection.

According to one embodiment herein, a recombinant vaccine composition against an urinary tract infection (UTI's) comprises of the following proteins: fimbrial protein and flagellin protein. The flagellin protein is in truncated form. The flagellin protein is taken in two truncated forms, and the two truncated forms of flagellin protein are a tFliCA and a tFliCB.

According to one embodiment herein, the fimbrial protein is a FimH protein. The fimbrial protein is taken from an uropathogenic Escherichia coli.

According to one embodiment herein, the first truncated form of flagellin protein is tFliCA, and wherein the first truncated form of flagellin comprises 79-117 amino acids. The second truncated form of flagellin protein is tFliCB, and the second truncated form of flagellin comprises 477-508 amino acids.

According to one embodiment herein, the first truncated form of flagellin tFliCA is placed at N terminal of the FimH protein. The second truncated form of flagellin tFliCB is placed at C terminal of the FimH protein.

According to one embodiment herein, the recombinant vaccine induces and stimulates a cellular immunity. The recombinant vaccine induces and stimulates a T helper cell 1 (Th1) response. The two forms of the T helper cell 1 respond to the recombinant vaccine. The two forms of T helper cell 1 are a Th1 form A and a Th1 form B. The recombinant vaccine induces and stimulates T helper cell 2 (Th2) response. T helper cell 2A respond to the recombinant vaccine. The recombinant vaccine induces a cellular immunity. The recombinant vaccine induces and stimulates a humoral immune response.

The recombinant vaccine induces and stimulates the production of interferon-γ (IFN-γ). The recombinant vaccine induces and stimulates the production of interleukin-4 (IL-4).

According to one embodiment herein, the recombinant vaccine interacts with a Toll like receptor-5 (TLR-5) and wherein an interaction free energy of the recombinant vaccine with the Toll like receptor-5 (TLR-5) is 935.0 kJ/mol. The composition has a size of the recombinant vaccine is 1200 bp. The recombinant vaccine induces and stimulates an innate immune response.

According to one embodiment herein, the method of synthesizing and testing the recombinant vaccine comprises the following steps fusion protein modeling, protein-protein interaction studies, isolation and procurement of bacterial strains, PCR amplification and cloning, expression and purification of recombinant protein, immunization of rats, evaluation of humoral immune response, cytokine secretion detection and statistical analysis.

According to one embodiment herein, in the fusion protein modeling the protein sequence of uropathogenic Escherichia coli FimH protein is obtained from NCBI data base. The sequences of tFliC are placed at the N and C termini of FimH protein to design different FimH/tFliC combinations. Modeling of fusion proteins is performed using I-Tasser server, a hierarchical modeling approach based on multiple threading alignment. The 3D structure of FimH, FliC and FimH/full length FliC fusions as the control group is also modeled. The modeled structures are validated and evaluated using Ramachandran Plot analysis (RAMPAGE).

According to one embodiment herein, for the protein protein interaction studies the tertiary structure of human TLR 5 is obtained from RCSB Protein Data Bank. The docking of the fusion proteins with TLR-5 is performed using Hex docking server. Total interaction free energies are calculated based on shape and electro-statistics.

According to one embodiment herein, the bacterial strains are procured from National Escherichia coli reference laboratory of Iran (NERL, Iran). The enteroaggregative Escherichia coli (EAEC) strain 042 and Uropathogenic Escherichia coli (UPEC) strain UTI89 are obtained from NERL. The expression host strains are obtained from Invitrogen.

According to one embodiment herein, in polymerase chain reaction (PCR) amplification and cloning the fusion protein is selected for cloning and expression. The amplification of tFliC A and tFliC B (two forms of fliC gene) of strain 042 is performed by PCR. The amplified fragments are cloned in frame with Glutathione-S-Transferase (GST) tag in pGEX5x-1 vector. The recombinant plasmids are transformed into E. coli and the proteins are expressed following induction with 1 mM IPTG. The recombinant proteins (GST-A, GST-B) are purified with GST fusion protein purification column (GeneScript). The DNA extraction and PCR amplification of fimH gene is also performed according to standard protocols. The construction of FimH-tFliC fusion protein is performed by overlap PCR.

The FimH-tFliCs fused DNA fragments are generated in two fusion steps: first the generation of truncA-FimH fusion followed by construction of truncA-FimH-truncB DNA coding sequence. The truncA and truncB are tFliC A and tFliC B respectively. Both steps are performed under the following conditions. Initial denaturation of DNA is done at 94° C. for 4 minutes followed by a cycle for overlapping of the primer fragments at 45° C. for 2 minutes. The overlapping of primers is followed by extension at 72° C. for 3 minutes. The PCR is set for 30 cycles of amplification with conditions set to [94° C. for 45 seconds, 55° C. for 45 seconds and 72° C. for 45 seconds and final elongation step at 72° C. for 10 minutes]. The PCR products are digested with BamH1 and XhoI restriction endonucleases. After restriction digestion the fragments are cloned in a plasmid with GST tag in pGEX-5X-1 vector and used for transformation of Escherichia coli cells. The fidelity of cloning is verified by restriction endonuclease digestion and DNA sequencing.

According to one embodiment herein, the Glutathione-S-transferase (GST) tagged recombinant protein i.e. GST-A-FimH-B is expressed in transformed bacterial strains. A and B are the truncated forms of tFliC protein i.e. tFliC A and tFliC B respectively. For the expression and purification of recombinant protein a single transformed colony is inoculated into 5 ml Luria Bertani (LB) broth comprising 100 μg/ml ampicillin and incubated overnight at 37° C. One ml of overnight culture is inoculated to 100 ml LB broth and cells are cultured in a shake flask incubator (OD600 of 0.5) at 37° C. The expression of proteins in the cells is induced for 4 hour with 0.2 mM isopropyl β-D-thiogalactopyranoside (IPTG). The protein expression is evaluated by electrophoresis on 12% sodium sodium dodecyl sulfate-polyacrylamide gel electrophoresis (SDS-PAGE). The protein expression is confirmed by Western blot analysis using horse raddish peroxidase (HRP) conjugated with goat anti GST antibody at a dilution of (1:1000). The fusion protein GST-A-FimH-B is purified using GST fusion protein purification column with a standard protocol. The lipopolysaccharide contamination is removed by treating the protein with Triton X114. The purified fusion protein GST-A-FimH-Bis quantified by standard protocol of Bradford protein assay.

According to one embodiment herein, after the fusion protein GST-A-FimH-B is purified the fusion protein is administered to mice for immunization. For the immunization process 4-5 week female BALB/c mice are provided by Pasteur Institute of Iran. The animals are handled in accordance with European community's council directive of 24 Nov. of 1986. Group of five mice (n=5) is subcutaneously immunized with 50 μg of the proteins namely GST protein, GST-A, GST-B and GST-A-Fim-B or phosphate buffer saline respectively. The mice are immunized on day 0, 14 and 28. Before each immunization the mice are bled and the serum is collected for the determination of serum antibodies.

According to one embodiment herein, after the mice are immunized with the fusion protein GST-A-Fim-B, the mice are evaluated for the humoral immune response. Total IgG, IgG1 and IgG2a antibody responses against the fusion protein GST-A-Fim-B is determined quantitatively. The antibody response is determined by quantitative enzyme linked immunosorbent assay (ELISA). The purified fusion protein GST-A-Fim-B is diluted in phosphate buffer saline (PBS) and 96-well plate is coated. Each well is coated with 5 μg. The plates are incubated overnight at 4° C. A 3% by volume bovine serum albumin (BSA) is added in the 96 well plate and the plate is incubated for 2 hours. After 2 hours the 96 well plate is washed with phosphate buffer saline (PBS) comprising 0.01% tween 20. The 96 well plate is incubated for 2 hours with 1/100 diluted serum. The next step is incubating the 96 well plate with HRP-conjugated goat anti-mouse IgG, IgG1 and IgG2a antibodies for one hour. After one hour the 96 well plate is washed and the plate is incubated with a substrate 3,3′,5,5′-Tetramethylbenzidine (TMB). The plates are subjected to absorbance of 450 nm and analyzed.

According to one embodiment herein, two weeks after last immunization the mice are tested for cytokine secretion detection. The mice spleens are removed and the cell suspensions are prepared by crushing the tissues through sterile grinder. The cell suspension is centrifuged at 1000 rpm for 10 minutes. The pellet is collected comprising the cells. A red blood cell lysis buffer is added to the pellet. The remaining cells are separated and re-suspended in Roswell Park Memorial Institute (RPMI) 1640 supplemented with 10% by volume fetal bovine serum (FBS) to obtain a suspension. The cell suspension is cultured in a 24-well plates for 72 hours at a density of 3×10⁵ in the presence of 10 μg/ml of each fusion protein (GST-A, GST-B and GST-A-Fim-B), Glutathion-S-transferase (GST) (negative control) or 5 μg/ml concanavalin A (ConA, positive control). Further the supernatant is collected and evaluated for secretion of INF-γ and IL-4 by mouse ELISA kits according to the standard protocol.

According to one embodiment herein, a statistical analysis is done for finding the differences between the mean values of the immunized groups are analyzed by ANOVA. The ANOVA analysis is followed by Tukey HSD test. The differences are considered statistically significant at a “p” values <0.05.

Six recombinant FimH/tFilC fusion variations are modeled using I-Tasser server via placing tFliC forms A and B at the N or C terminus of FimH. In addition, 3D structure of control group (FliC and FimH/full length FliC fusions) is also modeled.

The prediction of affinity of the modeled proteins to TLR-5 is investigated by considering parameters such as interaction free energy and pose of interaction. According to the results A-FimH-B (−935.0 kJ/mol) and FimH-A-B (−925.0 kJ/mol) showed high interaction tendency to TLR-5 according to the total free energy. B-FimH-A (−844.2 kJ/mol) and FimH-B-A (−816.6 kJ/mol) are the next best structures. Finally, B-A-FimH (−763.6 kJ/mol) and A-B-FimH (−771.4 kJ/mol) revealed the lowest total free energies. The docking conformations, in the A-FimH-B, B-A-FimH, FimH-A-B and FimH-B-A fusions, reveals that the truncated forms A and/or B directly interact with TLR-5 while in the other two fusions (B-FimH-A and A-B-FimH) direct interaction of truncated forms with TLR-5 is not possible. Comparison between the pose of interaction in forms A-FimH-B, FimH-A-B, B-A-FimH and FimH-B-A shows that in structures B-A-FimH, FimH-A-B and FimH-B-A only one of the truncated forms (A) effectively interact with TLR-5. Further in the A-FimH-B fusion, direct interaction of both forms (A and B) with receptor is possible. According to the results FimH-tFliC fusion proteins show lower total free energies when compared to the control group.

GST, GST-A and GST-B are constructed, expressed and purified. The fusion fragment is constructed by overlap PCR, digested and ligated with the similarly digested ends of pGEX-5X-1 plasmid. The cloning is verified by double digestion with BamHI and XhoI enzymes that confirm the cloning by releasing a fragment of about 1200 bp. The fidelity of the fusion fragment is confirmed by DNA sequencing. The fusion protein is expressed in E. coli and purified. The expression is confirmed by SDS-PAGE and Western blot analysis. Expression and purification of GST, GST-A and GST-B is also performed and confirmed as described earlier. The level of LPS in the protein preparations was ≦0.01 EU/ml and the proteins are used for immunization of mice.

After immunization, the antibody responses to GST-A, GST-B, GST-A-FimH-B (GST-fusion) and GST alone are determined by ELISA. Antigen specific IgG responses are detected after the first immunization in all groups compared to the negative control (PBS). Direct comparison of ELISA IgG responses to the recombinant proteins is not possible because different coating antigens are used on the solid phase of the ELISAs. The results illustrate that the GST tagged fusion proteins raise higher IgG responses compared to the GST (p<0.05) after the immunizations. The IgG response following stimulation with the GST-A-FimH-B is significantly higher than the other groups (p<0.02) after the first immunization indicating a strong immunogenicity.

The quality of the immune responses is evaluated. For the evaluation the IgG subclasses of the antigen-specific antibodies in serum samples are measured. Analysis of IgG subclasses revealed significantly higher IgG1 and IgG2a production in mice immunized with GST-A, GST-B and GST-A-FimH-B compared to PBS group (p<0.001). GST-A and GST-A-FimH-B significantly enhanced IgG1 and IgG2a responses over GST (p<0.01), while GST-B only raised higher IgG2a response compared to the GST group (p=0.006). The IgG1/IgG2a ratio dropped from 4.3 in mice immunized with GST to 3.8, 2.8 and 3 in mice immunized with GST-A, GST-B and GST-A-FimH-B, respectively.

Cytokine levels in immunized mice are evaluated by ELISA. All groups of mice showed higher IFN-γ and IL-4 levels (p<0.001) compared to the negative control phosphate buffer saline (PBS). The levels of INF-γ and IL-4 in mice immunized with GST-A is significantly higher compared to the GST group (p<0.001). The INF-γ level in mice immunized with GST-B is significantly higher than GST (p<0.001), while the IL-4 level is not different with the GST group. Higher levels of IFN-γ and IL-4 production is observed in mice immunized with GST-A-FimH-B (p<0.01). These results illustrate that all the three recombinant proteins (GST-A, B and A-FimH-B) are effective in stimulating T helper cell 1 (Th1) responses. Additionally, GST-A and GST-FimH-B are also capable of eliciting T-helper cell 2 (Th2) immune responses.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flowchart indicating a method for producing and analyzing the immunological response of recombinant protein GST-A-Fim-B, according to an embodiment herein.

FIG. 2 illustrates a table indicating Hex docking results for FimH-tFliC fusions, based on energy (E-total) and docking conformation, according to an embodiment herein.

FIG. 3 illustrates a schematic representation of the FliC A-FimH-FliC B fusion protein, according to one embodiment herein.

FIG. 4A and FIG. 4B illustrate the photographs indicating the gel electrophoresis illustrating the size of the recombinant protein DNA (FliC A-FimH-FliCB), according to one embodiment herein.

FIG. 5A and FIG. 5B illustrate the photographs indicating the Western blot analysis of the GST tagged FliCA-FimH-FliCB fusion protein, according to one embodiment herein.

FIG. 6A and FIG. 6B illustrates the graphs indicating the humoral immune responses induced by GST, GST-A, GST-B and GST-A-FimH-B (fusion protein), according to one embodiment herein.

FIG. 7A and FIG. 7B illustrates the graphs indicating the cytokine secretion induced by GST, GST-A, GST-B and GST-A-FimH-B (fusion protein), according to one embodiment herein.

Although the specific features of the embodiments herein are shown in some drawings and not in others. This is done for convenience only as each feature may be combined with any or all of the other features in accordance with the embodiments herein.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a recombinant vaccine against urinary tract infection. The recombinant vaccine consist of two truncated forms of flagellin (FliC) protein of enteroaggregative Escherichia coli placed at N and C termini of FimH protein derived from uropathogenic Escherichia coli. The recombinant vaccine is an adjuvant. Further the truncated sections of flagellin comprises 79-117 amino acids and 477-508 amino acids.

According to one embodiment herein, a recombinant vaccine composition against an urinary tract infection (UTI's) comprises of the following proteins: fimbrial protein and flagellin protein. The flagellin protein is in truncated form. The flagellin protein is taken in two truncated forms, and the two truncated forms of flagellin protein are a tFliCA and a tFliCB.

According to one embodiment herein, the fimbrial protein is a FimH protein. The fimbrial protein taken from an uropathogenic Escherichia coli.

According to one embodiment herein, the first truncated form of flagellin protein is tFliCA, and wherein the first truncated form of flagellin comprises 79-117 amino acids. The second truncated form of flagellin protein is tFliCB, and the second truncated form of flagellin comprises 477-508 amino acids.

According to one embodiment herein, the first truncated form of flagellin tFliCA is placed at the FimH protein. The second truncated form of flagellin tFliCB is placed at C terminal of the FimH protein.

According to one embodiment herein, the recombinant vaccine induces and stimulates a cellular immunity. The recombinant vaccine induces and stimulates a T helper cell 1 (Th1) response. The two forms of the T helper cell 1 respond to the recombinant vaccine. The two forms of T helper cell 1 are a Th1 form A and a Th1 form B. The recombinant vaccine induces and stimulates T helper cell 2 (Th2) response. T helper cell 2A respond to the recombinant vaccine. The recombinant vaccine induces a cellular immunity. The recombinant vaccine induces and stimulates a humoral immune response.

The recombinant vaccine induces and stimulates the production of interferon-γ (IFN-γ). The recombinant vaccine induces and stimulates the production of interleukin-4 (IL-4).

According to one embodiment herein, the recombinant vaccine interacts with a Toll like receptor-5 (TLR-5) and herein an interaction free energy of the recombinant vaccine with the Toll like receptor-5 (TLR-5) is 935.0 kJ/mol. The composition has a size of the recombinant vaccine is 1200 bp. The recombinant vaccine induces and stimulates an innate immune response.

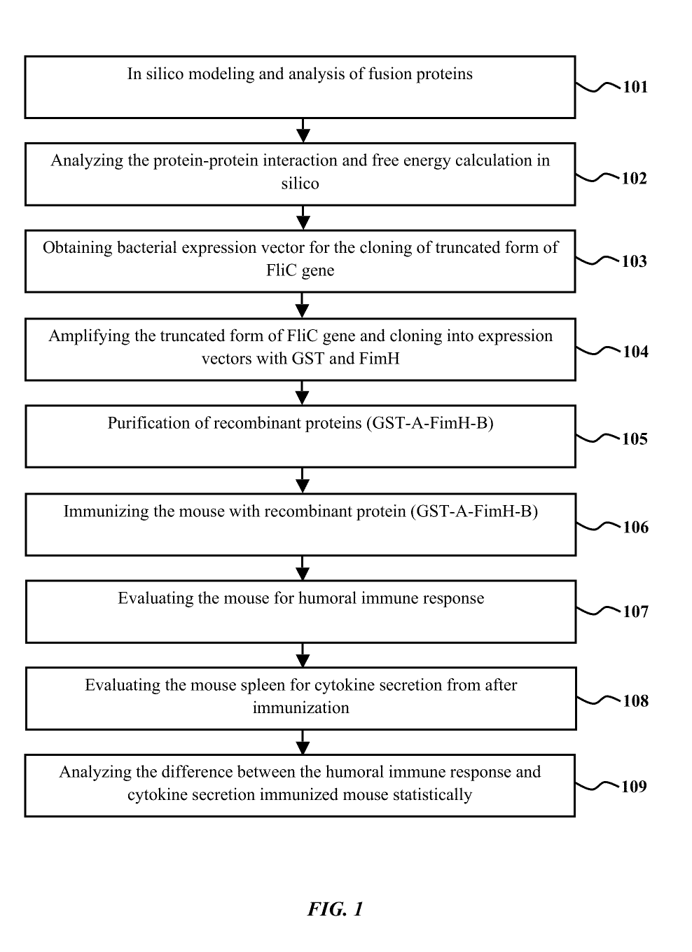

FIG. 1 illustrates a flowchart indicating a method for producing and analyzing the immunological response of recombinant protein GST-A-Fim-B, according to an embodiment herein. With respect to FIG. 1, the first step is in silico modeling and analysis of fusion proteins (101). The next step is analyzing the protein-protein interaction and free energy calculation in silico (102). Further obtaining bacterial expression vector for the cloning of truncated form of FliC gene (103). The next step is amplifying the truncated form of FliC gene and cloning into expression vectors with GST and FimH (104). The next step is purification of recombinant proteins (GST-A-Fim-B) (105). Further immunizing the mouse with recombinant protein (GST-A-FimH-B) (106). The mice are evaluated for the humoral immune response (107). The mouse spleen is analyzed for the cytokine secretion after immunization (108). The next step is analyzing the difference between the humoral immune response and cytokine secretion in immunized mouse statistically (109).

### Experimental Methods

### Experiment-1 Fusion Protein Modeling

Protein sequence of uropathogenic Escherichia coli FimH protein was obtained from NCBI data base (Genbank accession no. YP 543951.1). The sequence of tFliC forms were placed at the N and C termini of FimH protein to design different FimH/tFliC combinations. Modeling of fusion proteins was performed using I-Tasser server, a hierarchical modeling approach based on multiple threading alignment. 3D structure of FimH, FliC and FimH/full length FliC fusions as the control group was also modeled. The modeled structures were validated and evaluated using RAMPAGE and ProSa web.

### Experiment-2 Protein-Protein Interaction Studies

The tertiary structure of human TLR-5 was obtained from RCSB Protein Data Bank (PDB: 3J0A). Docking of fusion proteins with TLR-5 was performed using Hex docking server. Total interaction free energies were calculated based on shape and electrostatics as correlation type and final search was set to 25 (N=25). Other parameters were set to default values.

### Experiment-3 Obtaining Bacterial Strains

EAEC strain 042 and UPEC strain UTI89 was obtained from National Escherichia coli reference laboratory of Iran (NERL, Iran). The expression host, Top 10 strain of E. coli was obtained from Invitrogen.

### Experiment-4 PCR Amplification and Cloning

The fusion protein (GenBanck accession no. JX083850.1) was selected for cloning and expression. Amplification of tFliC forms A and B of fliC gene of strain 042 was performed by PCR and standard protocols. Amplified fragments are cloned in frame with GST tag in pGEX5x-1 vector. Recombinant plasmids are transformed into E. coli Top 10 and the proteins are expressed following induction with 1 mM IPTG. Recombinant proteins (GST-A, GST-B) are purified with GST fusion protein purification column (GeneScript). DNA extraction and PCR amplification of fimH gene was also performed according to standard protocol and construction of FimH-tFliCs fusion protein was performed by overlap PCR. All primer sets are listed in Table 1 below:

| Sequence (5′-3′) | Primer name |
|---|---|
| 5′ CCG GGA TCC AGG AAG GCG CGC | TruncA-Fr |
| TGT CCG 3′ | |
| 5′ AAC TCG TTT CAT TTT GAT TTC | TruncA(FimH)-Rv |
| GTC CTG G 3′ | |
| 5′ GAC GAA ATC AAA ATG AAA CGA | FimH(TruncA)-Fr |
| GTT ATT ACC 3′ | |
| 5′ GAATTTGTCGATTTGATAAACAAAAGT | FimH(truncB)-Rv |
| CACG 3′ | |
| 5′ TTT GTT TAT CAA ATC GAC AAA | TruncB(FimH)-Fr |
| TTC CGT TC 3′ | |
| 5′ CCG CTC GAG TCA CGC TTC AGA | TruncB-Rv |
| CAG GTT G 3′ | |

FimH-tFliCs fused DNA fragments were generated in two fusion steps: first, generation of truncA-FimH fusion followed by construction of truncA-FimH-truncB DNA coding sequence. Both steps were performed under the following conditions: initial denaturation at 94° C. for 4 min then a cycle for overlapping of the fragments at 45° C. for 2 min followed by extension at 72.0 for 3 min, then, 30 cycles of 94° C. for 45 s, 55.0 for 45 s and 72.0 for 45 s, and a final elongation step at 72° C. for 10 min. PCR products were digested with BamHI and XhoI restriction endonucleases, cloned in frame with GST tag inpGEX-5X-1 vector (GE Healthcare) and used for transformation of Top 10 E. coli cells. The fidelity of cloning was verified by restriction endonuclease digestion and DNA sequencing.

### Experiment-5 Expression and Purification of Recombinant Protein

Glutathion-S-transferase (GST) tagged recombinant protein (GST-A-FimH-B) was expressed. Briefly, a single transformed colony was inoculated into 5 ml LB broth containing 100 g/ml ampicillin and grown overnight at 37° C. One ml of overnight culture was inoculated to 100 ml LB broth and cells were cultured at 37° C. while shaking to an OD600 of 0.5 and the expression was induced for 4 h with 0.2 mM isopropyl-d-thiogalactopyranoside (IPTG). Protein expression was evaluated by electrophoresis on 12% SDS-PAGE and confirmed by Western blot analysis using HRP conjugated goat anti GST antibody (GenScript, USA) at a dilution of (1:1000). Fusion protein was purified using GST Fusion Protein Purification column (GeneScript, USA) according to the manufacturer's instructions. Lipopolysaccharide contamination was removed by Triton X114 (Sigma) by following standard protocol. The Limulus assay was used to determine the level of LPS contamination. Purified fusion protein was quantified by Bradford protein assay.

Experiment-6 Immunization of Mice with Recombinant Protein

The 4-5 weeks old female BALB/c mice were provided by Pasteur Institute of Iran. The animals were handled in accordance with European community's council directive of 24 Nov. of 1986 (86/609/EEC). Groups of five mice (n=5) were subcutaneously immunized with 50 g of the proteins (GST, GST-A, GST-B and GST-A-FimH-B) or PBS on days 0, 14 and 28. Before each immunization the mice were bled and sera were collected for the determination of serum antibodies.

### Experiment-7 Evaluation of Humoral Immune Response

Total IgG, IgG1 and IgG2a antibody responses against the GST tagged fusion proteins were determined quantitatively by enzyme-linked immunosorbent assay (ELISA). Briefly, purified GST-tagged fusion proteins were diluted in PBS and used to coat 96-well plates (5 g/well), and then the plates were incubated overnight at 4° C. After a blocking step of 2 h with 3% bovine serum albumin (BSA; Sigma), the wells were washed with PBS containing 0.01% tween20 and incubated for 2 h with 1/100 diluted sera. Afterwards, the plates were incubated with HRP-conjugated goat anti-mouse IgG, IgG1 and IgG2a (Sigma; USA) for 1 h prior, followed by washing, and incubation with substrate (TMB). Finally, the plates were subjected to absorbance read at 450 nm.

### Experiment-8 Cytokine Secretion Detection

Two weeks after last immunization, mice spleens were removed and cell suspensions prepared by crushing the tissues through sterile grinder. Cells were then pelleted by centrifugation (1000 rpm, 10 min) and red blood cell lysis buffer was added. Then the remaining cells were separated and resuspended in RPMI 1640 (Gibco; USA) supplemented with 10% fetal bovine serum (FBS) and cultured in 24-well plates for 72 h at a density of 3×10⁵ in the presence of 10 g/ml of each fusion proteins, GST (negative control) or 5 g/ml concanavalin A (ConA, positive control). Then, cell culture supernatants were collected and evaluated for secretion of INF-γ and IL-4 by Mouse ELISA kits (Abcam) according to the manufacturer's protocol.

### Experiment-8 Statistical Analysis

The differences between the mean values of the immunized groups were analyzed by ANOVA followed by Tukey HSD test. Differences were considered statistically significant at p values <0.05.

### Results

### Result-1 Fusion Protein Modeling

Six recombinant FimH/tFilC fusion variations were modeled using I-Tasser server via placing tFliC forms A and B at the N or C terminus of FimH. In addition, 3D structure of control group (FliC and FimH/full length FliC fusions) was also modeled. Model evaluation was performed using ProSa-(Protein structure analysis) web and Rampage. The Ramachandran plots of modeled structures revealed that, 95, 96.5, 95.4, 93, 97.2, and 96.5% of residues fell within the allowed regions according to the fusion proteins: A-FimH-B, B-FimH-A, A-B-FimH, B-A-FimH, FimH-A-Band FimH-B-A, respectively. Additionally, validation of 3-D structures with ProSa-web revealed that all Z-score values were within the range of native conformations of crystal structures, indicating the good quality of the models (Supplementary data).

### Result-2 Docking Analysis

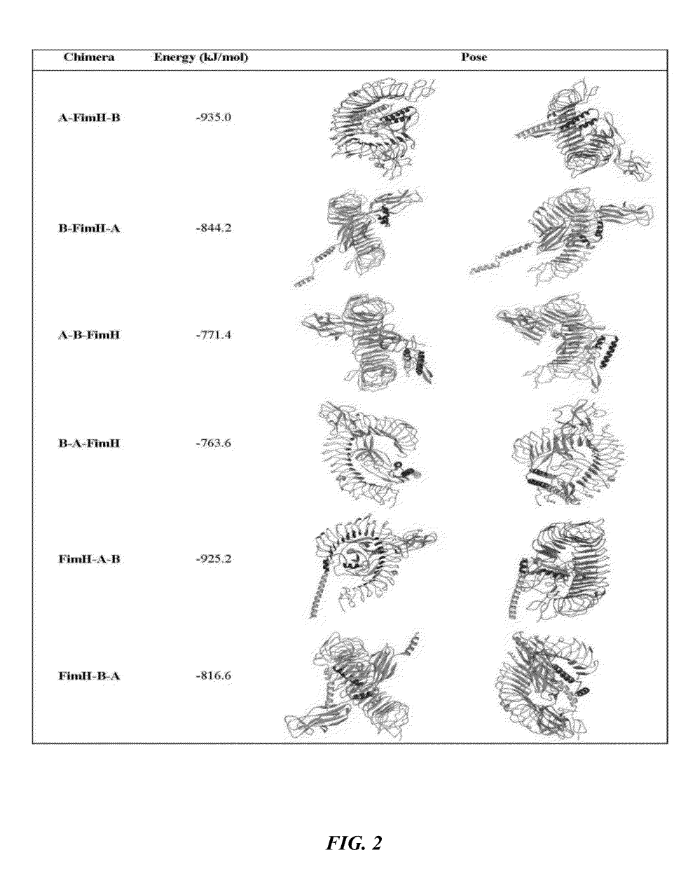

FIG. 2 illustrates a table indicating Hex docking results for FimH-tFliC fusions, based on energy (E-total) and docking conformation, according to an embodiment herein. The prediction of affinity of the modeled proteins to TLR-5 is investigated by considering parameters such as interaction free energy and pose of interaction. According to the table, A-FimH-B (−935.0 kJ/mol) and FimH-A-B (−925.0 kJ/mol) showed the best interaction tendency to TLR-5 according to the total free energy. B-FimH-A (−844.2 kJ/mol) and FimH-B-A (−816.6 kJ/mol) were the next best structures. Finally, B-A-FimH (−763.6 kJ/mol) and A-B-FimH (−771.4 kJ/mol) revealed the worst total free energies. Considering docking conformations, in the A-FimH-B, B-A-FimH, FimH-A-B and FimH-B-A fusions, the truncated forms A and/or B can directly interact with TLR-5 while in the other two fusions (B-FimH-A and A-B-FimH) direct interaction of truncated forms with TLR-5 was not possible. Comparison between the pose of interaction in forms A-FimH-B, FimH-A-B, B-A-FimH and FimH-B-A showed that in structures B-A-FimH, FimH-A-B and FimH-B-A only one of the truncated forms (A) can effectively inter-act with TLR-5 while in the A-FimH-B fusion, direct interaction of both forms (A and B) with receptor was possible.

Table 2 below lists the docking results of the control group (FliC, native FliC-FimH and FimH-native FliC) performed by Hex.

| Docking complex | Total energy (kj/mol) |
|---|---|
| FliC/TLR-5 | −716 |
| FimH-FliC/TLR-5 | −366.7 |
| FliC-FimH/TLR-5 | −735.6 |

According to FIG. 2 and Table 2 FimH-tFliC fusion proteins showed lower total free energies compared to the control group.

### Result-3 Cloning, Expression and Purification of Recombinant Proteins

GST, GST-A and GST-B are constructed, expressed and purified as described in methods.

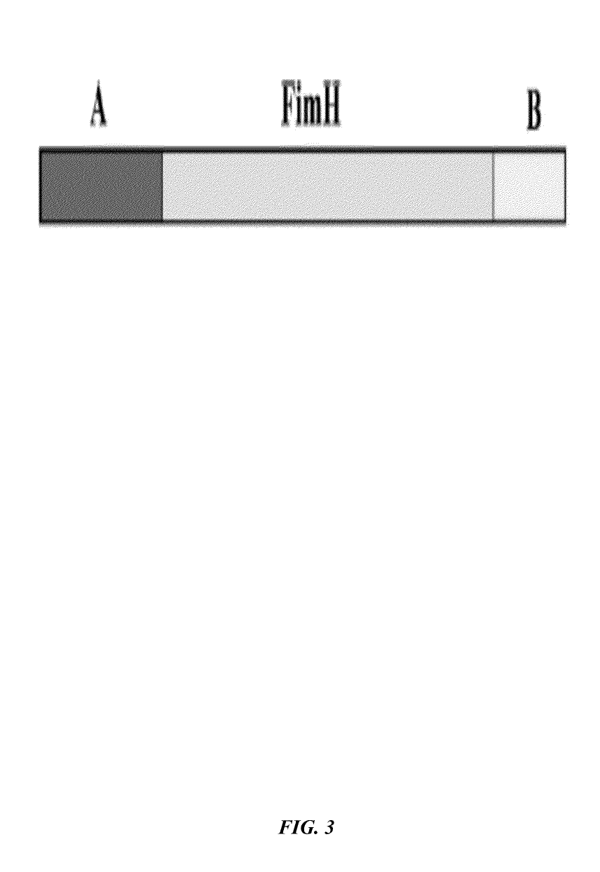

FIG. 3 illustrates a schematic representation of the FliC A-FimH-FliCB fusion protein, according to one embodiment herein. The fusion fragment is constructed by overlap PCR digested and ligated with the similarly digested ends of pGEX-5X-1 plasmid. The truncated protein FliC A and FliC B are fused to 3′ and 5′ terminus of the fimH coding sequence.

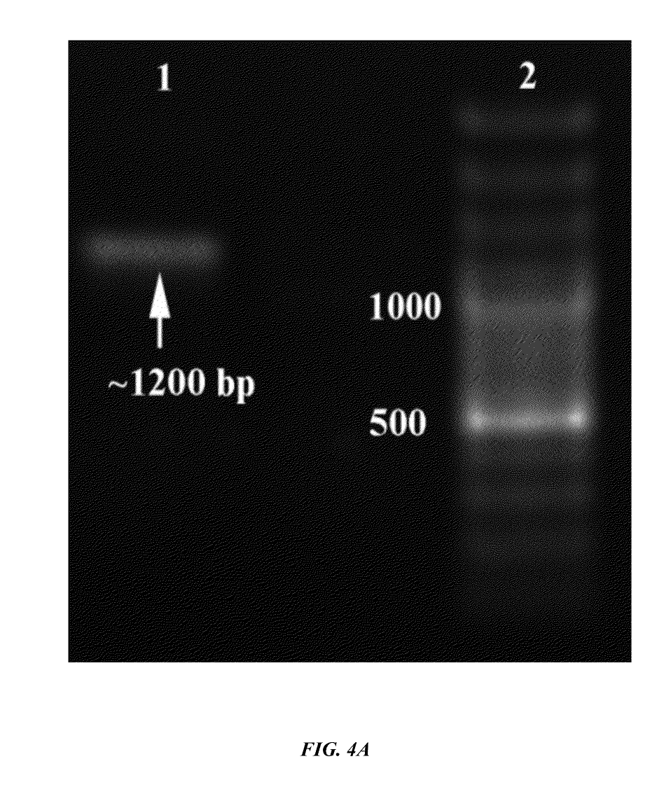

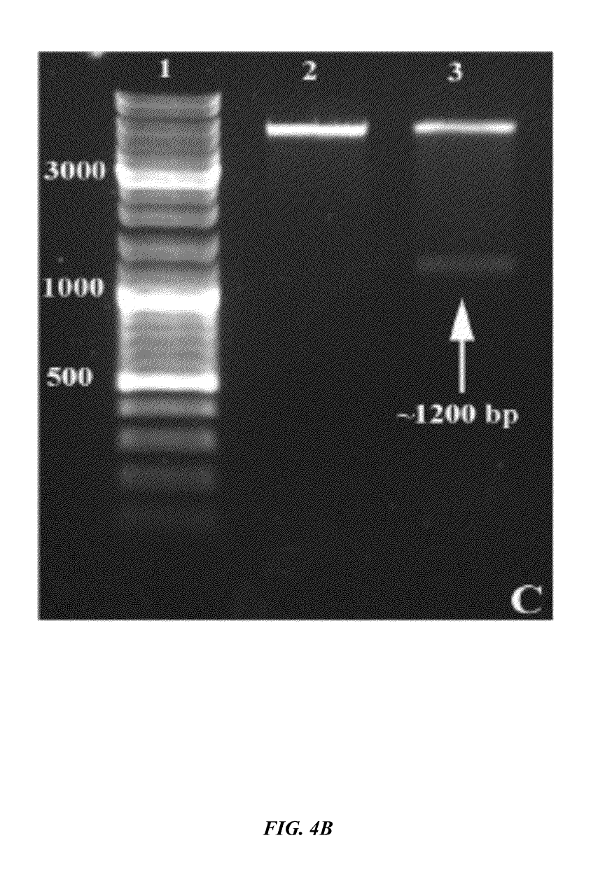

FIG. 4A and FIG. 4B are the photographs indicating the gel electrophoresis illustrating the size of the recombinant protein DNA (FliC A-FimH-FliCB), according to one embodiment herein. The cloning of FliC A-FimH-FliCB fusion protein in the plasmid is verified by double digesting the plasmid with BamHI and XhoI enzymes. The cloning is confirmed with the presence of a DNA fragment 1200 bp, as illustrated in FIG. 4A and FIG. 4B. FIG. 4A illustrates the PCR products of FliC A-FimH-FliCB fusion protein sequence. The photograph of gel in the FIG. 4A illustrates lane 1 of the gel loaded with DNA marker and lane 2 of the gel loaded with fusion fragment. FIG. 4B illustrates the results of the double digestion of recombinant plasmid. The lane 1 of the gel is loaded with DNA marker, lane 2 of the gel is loaded with the digested non-recombinant PGEX-5X-1 plasmid and the lane 3 of the gel is loaded with the digested p-GEX-A-FimH-B plasmid confirming the cloning procedure. The gel illustrates a DNA band of 1200 bp. The fidelity of the fusion fragment or DNA fragment of FliCA-FimH-FliC is confirmed by DNA sequencing. The fusion protein is expressed in E. coli and purified.

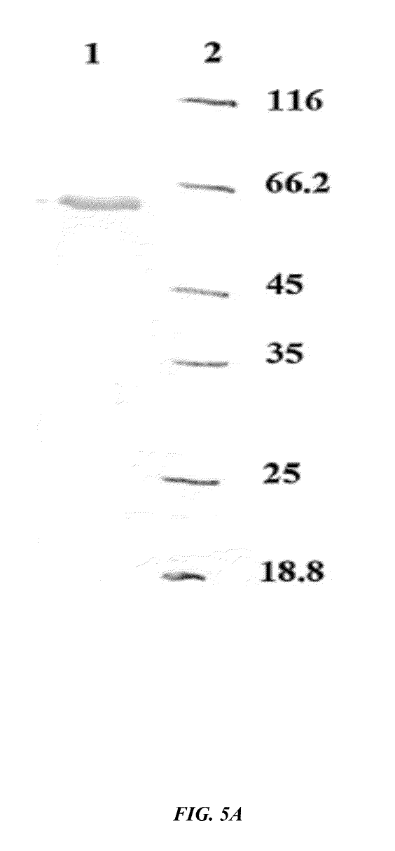

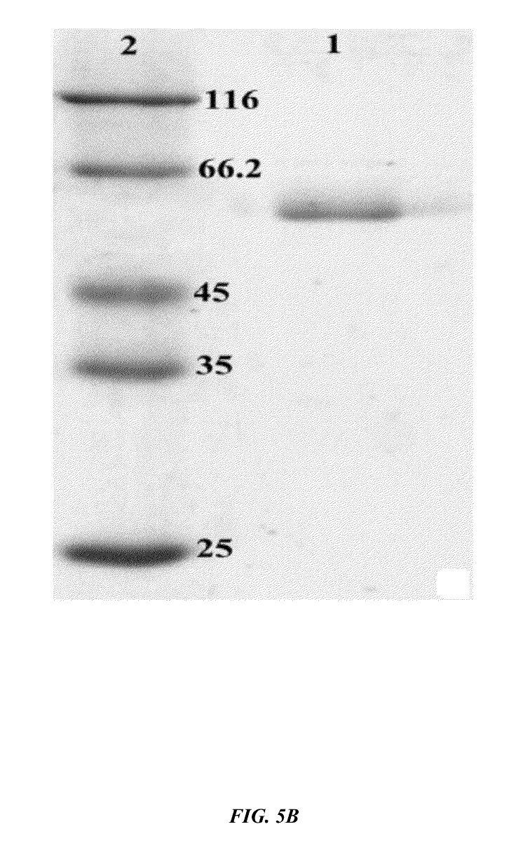

FIG. 5A and FIG. 5B are the photographs indicating the Western blot analysis of the GST tagged FliCA-FimH-FliCB fusion protein, according to one embodiment herein. The expression of the fusion protein is confirmed by SDS-PAGE and Western blot analysis. In FIG. 5A and FIG. 5B the lnne 1 is loaded with the FliCA-FimH-FliCB fusion protein. The lane 2 is loaded with the Expression and purification of GST, GST-A and GST-B is also performed and confirmed as per standard protocol. The level of LPS in the protein preparations is <0.01 EU/ml and the proteins are used for immunization of mice.

### Result-4 Humoral Immune Responses to the Recombinant Proteins

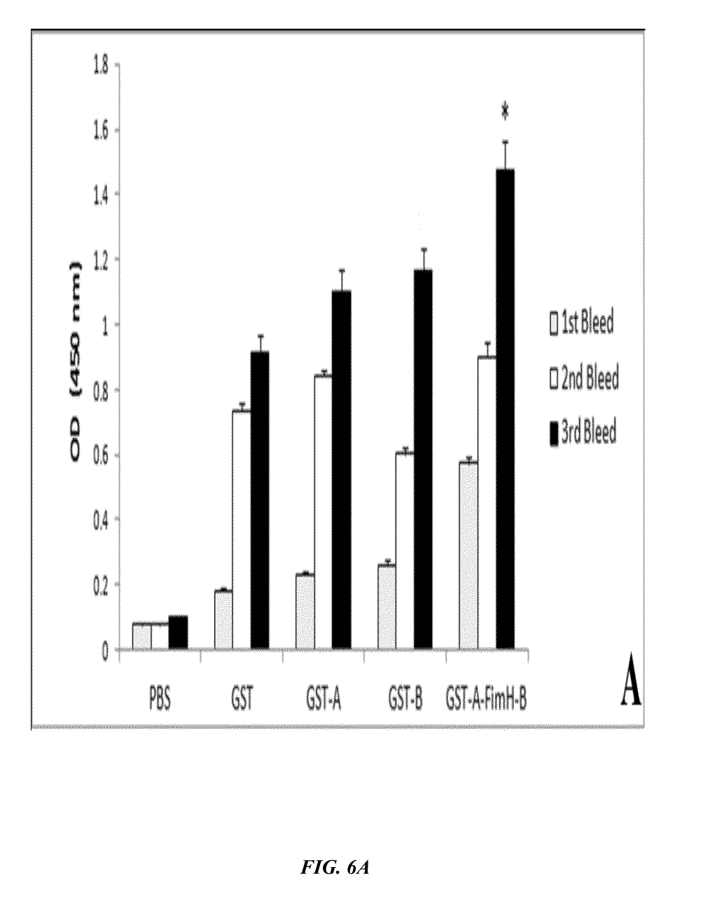

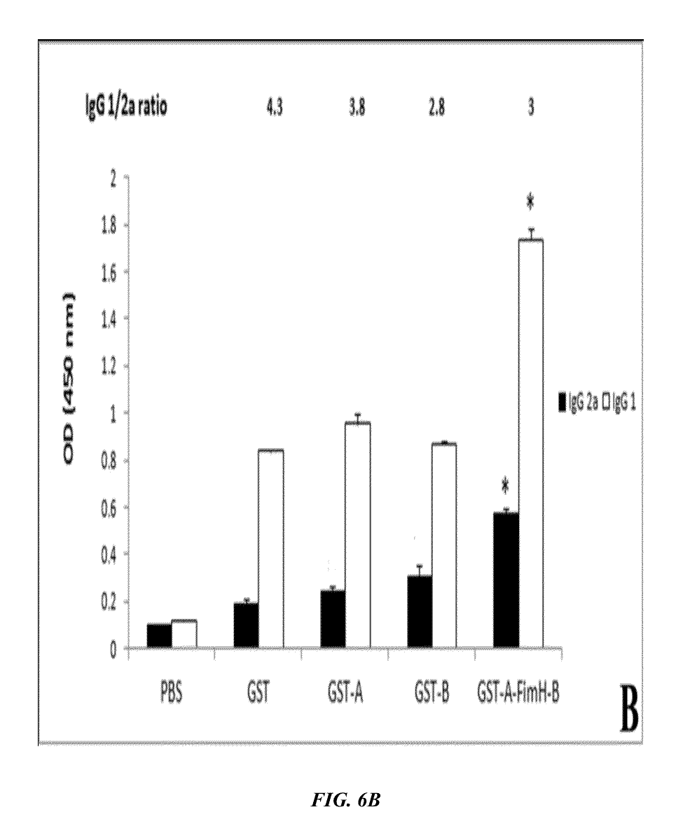

After immunization, the antibody responses to GST-A, GST-B, GST-A-FimH-B (GST-fusion) and GST are determined by ELISA. FIG. 6A and FIG. 6B illustrates the graphs indicating the humoral immune responses induced by GST, GST-A, GST-B and GST-A-FimH-B (fusion protein), according to one embodiment herein. FIG. 6A illustrates total IgG antibody response in immunized mice after each bleed. FIG. 6A also illustrates the detecting the antigen specific IgG responses after the first immunization in all groups compared to the negative control (PBS). Direct comparison of ELISA IgG responses to the recombinant proteins is not possible because different coating antigens are used on the solid phase of the ELISAs. The results suggest that the GST tagged fusion proteins raise higher IgG responses compared to the GST (p<0.05) after the immunizations. The IgG response following stimulation with the GST-A-FimH-B is significantly higher than the other groups (p<0.02) after the first immunization indicating high immunogenicity and humoral response.

For evaluation of the quality of the immune responses, the IgG subclasses of the antigen-specific antibodies in serum samples are measured. FIG. 6B illustrates the analysis of IgG subclasses. Further the FIG. 6B illustrates that the production of the IgG subclasses IgG1 and IgG2a is significantly higher in mice immunized with GST-A, GST-B and GST-A-FimH-B compared to PBS group (p<0.001). GST-A and GST-A-FimH-B significantly enhanced IgG1 and IgG2a responses over GST (p<0.01), while GST-B only raised higher IgG2a response compared to the GST group (p=0.006). The IgG1/IgG2a ratio dropped from 4.3 in mice immunized with GST to 3.8, 2.8 and 3 in mice immunized with GST-A, GST-B and GST-A-FimH-B, respectively.

### Result-5 Cloning, Expression and Purification of Recombinant Proteins

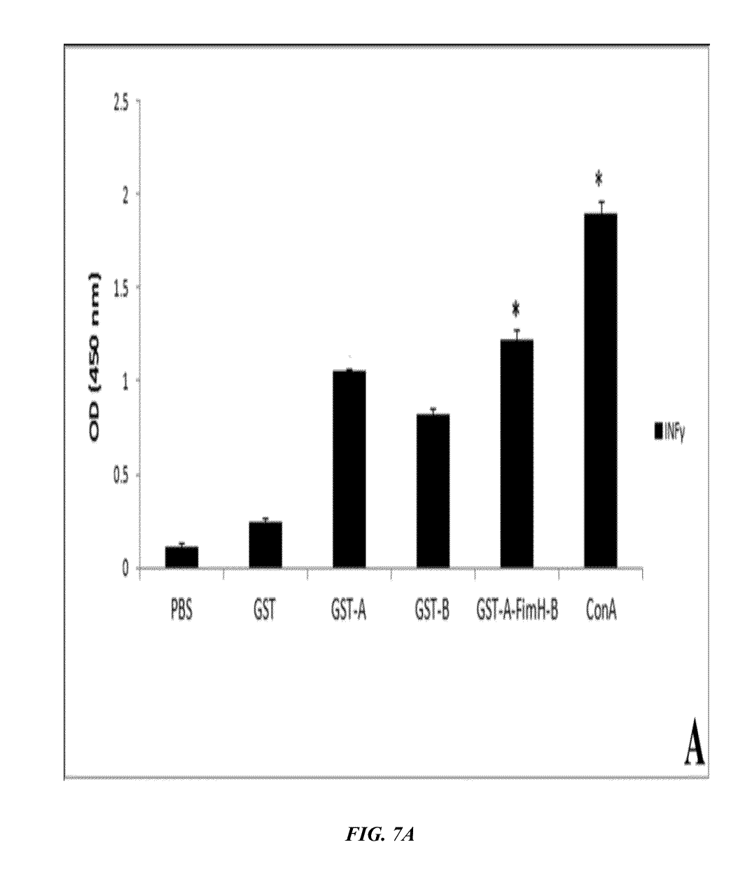

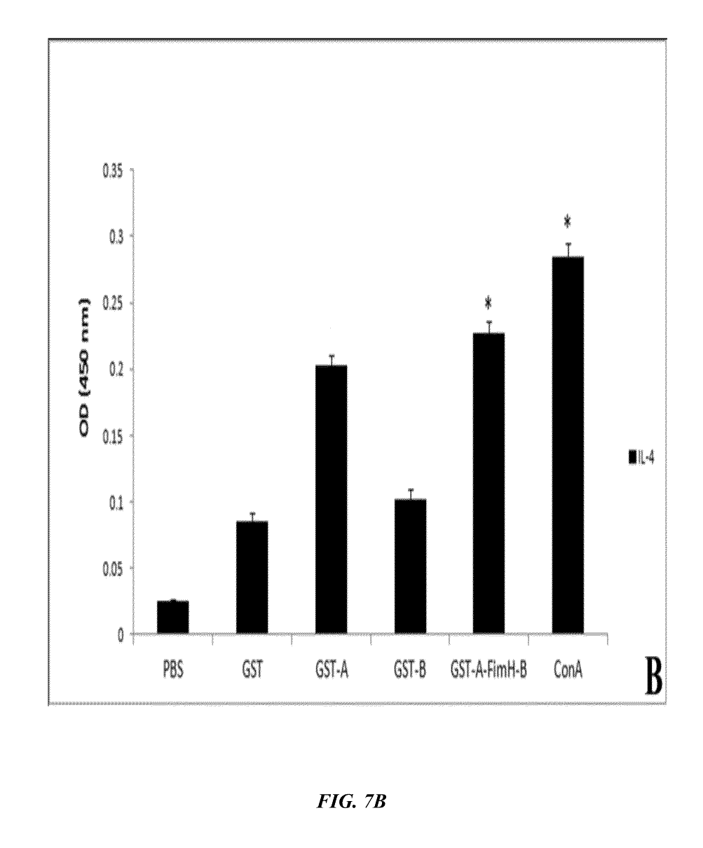

FIG. 7A and FIG. 7B illustrates the graphs indicating the cytokine secretion induced by GST, GST-A, GST-B and GST-A-FimH-B (fusion protein), according to one embodiment herein. Cytokine levels in immunized mice are evaluated by ELISA. As illustrated by FIG. 7A and FIG. 7B, all groups show a higher IFN-γ and IL-4 levels (p<0.001) compared to the negative control (PBS). The levels of INF-γ and IL-4 in mice immunized with GST-A is significantly higher compared to the GST group (p<0.001). The INF-γ level in mice immunized with GST-B is significantly higher than GST (p<0.001), while the IL-4 level is not different with the GST group. However, higher levels of IFN-γ and IL-4 production is observed in mice immunized with GST-A-FimH-B (p<0.01). These results also illustrate that all the three recombinant proteins (GST-A, GST-B and A-FimH-B fusion protein) are effective in stimulating T helper1 (Th1) responses. Additionally, GST-A and GST-FimH-B are also capable of eliciting Th2 immune responses.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A recombinant vaccine composition against an urinary tract infection (UTI's) comprises:
A fimbrial protein; and
A flagellin protein;
wherein the flagellin protein is in truncated forms, and wherein the flagellin protein is taken in two truncated forms, and wherein the two truncated forms of flagellin protein are a tFliCA and a tFliCB.

2. The composition according to claim 1, wherein the fimbrial protein is a FimH protein.

3. The composition according to claim 1, wherein the fimbrial protein is taken from an uropathogenic Escherichia coli.

4. The composition according to claim 1, wherein the first truncated form of flagellin protein is tFliCA, and wherein the first truncated form of flagellin comprises 79-117 amino acids.

5. The composition according to claim 1, wherein the second truncated form of flagellin protein is tFliCB, and wherein the second truncated form of flagellin comprises 477-508 amino acids.

6. The composition according to claim 1, wherein the first truncated form of flagellan tFliCA is placed at N terminal of the FimH protein.

7. The composition according to claim 1, wherein the second truncated form of flagellin tFliCB is placed at C terminal of the FimH protein.

8. The composition according to claim 1, wherein the recombinant vaccine induces and stimulates a cellular immunity.

9. The composition according to claim 1, wherein the recombinant vaccine induces and stimulates a T helper cell 1 (Th1) response and wherein the two forms of the T helper cell 1 respond to the recombinant vaccine, and wherein the two forms of T helper cell 1 are a Th1 form A and a Th1 form B.

10. The composition according to claim 1, wherein the recombinant vaccine induces and stimulates T helper cell 2 (Th2) response and wherein a T helper cell 2 A respond to the recombinant vaccine.

11. The composition according to claim 1, wherein the recombinant vaccine induces a cellular immunity.

12. The composition according to claim 1, wherein the recombinant vaccine induces and stimulates a humoral immune response.

13. The composition according to claim 1, wherein the recombinant vaccine induces and stimulates the production of interferon-γ (IFN-γ).

14. The composition according to claim 1, wherein the recombinant vaccine induces and stimulates the production of interleukin-4 (IL-4).

15. The composition according to claim 1, wherein the recombinant vaccine interacts with a Toll like receptor-5 (TLR-5) and wherein an interaction free energy of the recombinant vaccine with the Toll like receptor-5 (TLR-5) is 935.0 kJ/mol.

16. The composition according to claim 1, wherein the composition has a size of the recombinant vaccine is 1200 bp.

17. The composition according to claim 1, wherein the recombinant vaccine induces and stimulates an innate immune response.
